(19) United States
(12) Patent Application Publication
(10) Pub. No.: US 2019/0275119 A1
(43) Pub. Date: Sep. 12, 2019

(54) PROOXIDANT CANCER CHEMO-SUPPRESSORS AND CHEMO-PROTECTORS AND METHODS OF USE RELATED THERETO

(71) Applicant: Randolph M Howes, Kentwood, LA (US)

(72) Inventor: Randolph M Howes, Kentwood, LA (US)

(21) Appl. No.: 16/348,077

(22) PCT Filed: Nov. 7, 2018
(86) PCT No.: PCT/US2017/060343

§ 371 Date: May 7, 2019

Related U.S. Application Data
(60) Provisional application No. 62/418,476, filed on Nov. 7, 2016.

(51) Int. Cl. A61K 38/44; A61K 31/366; A61K 36/185; A61K 31/593; A61K 36/9066; A61K 31/26; A61K 31/11; A61K 36/88; A61K 31/385; A61K 33/40

(52) U.S. Cl. CPC A61K 38/446; A61K 31/366; C12Y 115/01001; A61K 38/443; C12Y 101/03004; A61K 36/185; A61K 33/40; A61K 36/9066; A61K 31/26; A61K 31/11; A61K 36/88; A61K 31/385; A61K 31/593

(57) ABSTRACT

Formulation(s) of prooxidation agents and/or antioxidant capacity reducing agents for producing electronically modified oxygen derivatives (“EMODs”) for cancer chemo-suppression and chemo-protection, and kit(s) and method(s) of use thereof.

### CROSS REFERENCE TO RELATED APPLICATIONS

The present application claims the benefit under 35 U.S.C. 119(e) of U.S. Provisional Application No. 62/418,476, filed Nov. 7, 2016, the entirety of which is hereby expressly incorporated herein by reference.

### STATEMENT REGARDING FEDERALLY FUNDED RESEARCH OR DEVELOPMENT

Not Applicable.

### TECHNICAL FIELD

The presently disclosed and claimed inventive concept(s) relate to formulations and methods of administration for cancer chemo-suppression and chemo-protection. More specifically, the presently disclosed and claimed inventive concept(s) relate to nutraceutical formulations comprising a plurality of prooxidant agents and/or antioxidant capacity reducing agents that are capable of activating the apoptotic process for the killing and/or protection against cancer cells as well as methods of use related thereto.

### BACKGROUND

Cancer is a general term used to indicate any of the various types of abnormal tissues that grow by cellular proliferation more rapidly than normal cells. Not only do cancer cells proliferate at a rate that is higher than normal cells but they are also likely to recur after attempted removal and cause death unless adequately suppressed. As such, cancer is one of the top health conditions facing populations today.

Numerous pharmaceuticals and methods exist for cancer treatment. The primary methods and compositions of treatment are chemotherapy and radiation therapy. At best, such drug-targeted therapies are limited in their effectiveness; at worst, they are rife with serious side effects and morbidity. Both chemotherapy and radiation can severely decrease a patient's immune system, increase cancer proliferation, and destroy the patient's oxidative ability to suppress, prohibit, and, in some instances, eradicate, cancer. Both chemotherapy and radiation are also extremely costly.

Accordingly, a need exists for formulation(s), kit(s), and rnethod(s) of cancer protection and suppression that have minimal side effects and are cost-effective. Such treatment can be used prophylactically to reduce the incidence of cancer and as an aid in combination with any existing cancer therapies. It is to such formulation(s), kit(s), and method(s) that the currently disclosed and/or claimed inventive concept(s) is/are directed.

Some believe that cancer is, at least in part, either caused or exacerbated by foods having high oxidation levels. For example, it has been suggested that the oxygen and radicals found in electronically modified oxygen derivatives (“EMODs”; formerly called reactive oxygen species or “ROS” or oxygen free radicals) induce cancer formation. Contrary to perpetual teachings in the prior art, not only do EMODs not increase a patient's risk of cancer but, when maintained at a critical level, they are highly effective in inhibiting cancer.

EMODs carry out highly sophisticated intra-, inter-, and extra- cellular signaling roles that are essential for normal biochemical functioning. The human body is capable of eradicating itself of cancer cells through EMOD-induced apoptosis. Apoptosis, or cellular suicide, is one of the most important means of eliminating precancerous and cancerous cells from the body. Cell apoptosis is initiated by extracellular and intracellular signals, such as the signals carried out by EMODs. Thus, studies show that EMODs may be major participants in inducing and triggering the apoptotic cascade resulting in cancer-cell death.

For self-protection, cancer cells produce excess lactic acid, an antioxidant, as they ferment energy. Lactic acid is toxic and tends to prevent the transport of oxygen into neighboring normal cells, a mechanism which counteracts EMODs' functions and efficacy. The selective concentration of antioxidants allows cancerous cells to protect themselves from EMOD-induced apoptotic death. Disruption of the balance between prooxidants and antioxidants has been implicated in the pathophysiology of many chronic diseases, such as atherosclerosis, cancer, diabetes, strokes, arthritis and cataract formation. More specifically, various pathologies can result from oxidative stress-induced apoptotic signaling that is consequent to ROS increases and/or antioxidant decreases.

Even small amounts of excess molecular antioxidants or antioxidant enzymes can serve to block or negate EMOD-induced messengers that trigger an apoptotic cascade. If a patient consumes excessive amounts of antioxidants, such increased consumption can (although counterintuitive) promote cancerous growth and spread. Thus, contrary to prior theories and conjectures, excess antioxidant levels are undesirable because they can disrupt the balance between prooxidants and antioxidants, thereby facilitating the proliferation of cancer-cells.

This protective mechanism in which cancerous cells produce excess antioxidants results in a deficiency of EMODs. In this state of insufficient EMODs, damage to the nuclear DNA occurs and mutations prevail. Over time, as the cancer cells replicate, the cancer may spread if not destroyed by the immune system and other sources of EMODs.

Accordingly, a need exists for new and improved methods of restoring the oxidative EMOD abilities of a patient's immune system that has minimal side effects and is cost-effective. Such treatment thereby allows, by way of example and not by way of limitation, for protection and suppression of cancer cell proliferation. It is to such treatments, as well as formulations related thereto, that the presently disclosed and claimed inventive concepts are directed. The presently claimed inventive concepts include, but are not limited to, a prooxidant EMOD approach, which effectively increases the potential to diminish therapeutically all types of cancer because it exploits the one weakness that is common to every cancer cell: a higher EMOD level than normal cells and therefore, a lower threshold needed to trigger a death pathway.

### DETAILED DESCRIPTION

Before explaining at least one embodiment of the inventive concept(s) in detail by way of exemplary drawings, experimentation, results, and laboratory procedures, it is to be understood that the inventive concept(s) is not limited in its application to the details of construction and the arrangement of the components set forth in the following description or illustrated in the drawings, experimentation and/or results. The inventive concept(s) is capable of other embodiments or of being practiced or carried out in various ways. As such, the language used herein is intended to be given the broadest possible scope and meaning; and the embodiments are meant to be exemplary-not exhaustive. Also, it is to be understood that the phraseology and terminology employed herein is for the purpose of description and should not be regarded as limiting.

Unless otherwise defined herein, scientific and technical terms used in connection with the presently disclosed and claimed inventive concept(s) shall have the meanings that are commonly understood by those of ordinary skill in the art. Further, unless otherwise required by context, singular terms shall include pluralities and plural terms shall include the singular. The foregoing techniques and procedures are generally performed according to conventional methods well known in the art and as described in various general and more specific references that are cited and discussed throughout the present specification. The nomenclatures utilized in connection with, and the laboratory procedures and techniques of, analytical chemistry, synthetic organic chemistry, and medicinal and pharmaceutical chemistry described herein are those well-known and commonly used in the art.

All patents, published patent applications, and non-patent publications mentioned in the specification are indicative of the level of skill of those skilled in the art to which this presently disclosed and claimed inventive concept(s) pertains. All patents, published patent applications, and non-patent publications referenced in any portion of this application are herein expressly incorporated by reference in their entirety to the same extent as if each individual patent or publication was specifically and individually indicated to be incorporated by reference.

All of the devices, kits, and/or methods disclosed and claimed herein can be made and executed without undue experimentation in light of the present disclosure. While the compositions and methods of this presently disclosed and claimed inventive concept(s) have been described in terms of preferred embodiments, it will be apparent to those of skill in the art that variations may be applied to the compositions and/or methods and in the steps or in the sequence of steps of the method described herein without departing from the concept, spirit and scope of the presently disclosed and claimed inventive concept(s). All such similar substitutes and modifications apparent to those skilled in the art are deemed to be within the spirit, scope and concept of the inventive concept(s) as defined by the appended claims.

As utilized in accordance with the present disclosure, the following terms, unless otherwise indicated, shall be understood to have the following meanings:

The use of the word “a” or “an” when used in conjunction with the term “comprising” in the claims and/or the specification may mean “one,” but it is also consistent with the meaning of “one or more,” “at least one,” and “one or more than one.” The singular forms “a,” “an,” and “the” include plural referents unless the context clearly indicates otherwise. Thus, for example, reference to “a compound” may refer to 1 or more, 2 or more, 3 or more, 4 or more or greater numbers of compounds. The term “plurality” refers to “two or more.” The use of the term “or” in the claims is used to mean “and/or” unless explicitly indicated to refer to alternatives only or the alternatives are mutually exclusive, although the disclosure supports a definition that refers to only alternatives and “and/or.” Throughout this application, the term “about” is used to indicate that a value includes the inherent variation of error for the device, the method being employed to determine the value, or the variation that exists among the study subjects. For example but not by way of limitation, when the term “about” is utilized, the designated value may vary by ±20% or ±10%, or ±5%, or ±1%, or ±0.1% from the specified value, as such variations are appropriate to perform the disclosed methods and as understood by persons having ordinary skill in the art, The use of the term “at least one” will be understood to include one as well as any quantity more than one, including but not limited to, 2, 3, 4. 5, 10,15, 20, 30, 40, 50, 100, etc. The term “at least one” may extend up to 100 or 1000 or more, depending on the term to which it is attached; in addition, the quantities of 100/1000 are not to be considered limiting, as higher limits may also produce satisfactory results. In addition, the use of the term “at least one of X, Y and Z” will be understood to include X alone, Y alone, and Z alone, as well as any combination of X, Y and Z. The use of ordinal number terminology (i.e., “first”, “second”, “third”, “fourth”, etc.) is solely for the purpose of differentiating between two or more items and is not meant to imply any sequence or order or importance to one item over another or any order of addition, for example.

As used in this specification and claim(s), the terms “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

The term “or combinations thereof” as used herein refers to all permutations and combinations of the listed items preceding the term. For example, “A, B, C, or combinations thereof” is intended to include at least one of: A, B, C, AB, AC, BC, or ABC, and if order is important in a particular context, also BA, CA, CB, CBA, BCA, ACB, BAC, or CAB. Continuing with this example, expressly included are combinations that contain repeats of one or more item or term, such as BB, AAA, AAB, BBC, AAABCCCC, CBBAAA, CABABB, and so forth. The skilled artisan will understand that typically there is no limit on the number of items or terms in any combination, unless otherwise apparent from the context.

As used herein, the term “substantially” means that the subsequently described event or circumstance completely occurs or that the subsequently described event or circumstance occurs to a great extent or degree. For example, the term “substantially” means that the subsequently described event or circumstance occurs at least 90% of the time, or at least 95% of the time, or at least 98% of the time.

As used herein, the phrase “associated with” includes both direct association of two moieties to one another as well as indirect association of two moieties to one another. Non-limiting examples of associations include covalent binding of one moiety to another moiety either by a direct bond or through a spacer group, non-covalent binding of one moiety to another moiety either directly or by means of specific binding pair members bound to the moieties, incorporation of one moiety into another moiety such as by dissolving one moiety in another moiety or by synthesis, and coating one moiety on another moiety.

The term “additive,” as used herein, means that the combination of the various agents and/or compounds are more effective in accomplishing the presently disclosed and/or claimed inventive concept(s) than an individual agent and/or compound.

The term “patient” includes human and veterinary subjects. In certain embodiments, a patient is a mammal. In certain other embodiments, the patient is a human. “Mammal” for purposes of treatment refers to any animal classified as a mammal, including human, domestic and farm animals, nonhuman primates, and zoo, sports, or pet animals, such as dogs, horses, cats, cows, etc.

The presently disclosed and/or claimed inventive concept(s) are primarily directed to novel biochemical formulation(s), kit(s), and methods) for diminishing a benign or malignant tumor in a patient. In one aspect of the inventive concept(s), the novel biochemical formulation(s) comprise biochemical prooxidant agents and/or antioxidant capacity reducing agents, both of which are individually or collectively capable of generating apoptotic-triggering electronically modified oxygen derivatives (“EMOD” or “EMODs”), formerly referred to in the literature as reactive oxygen species (“ROS”). Additionally, the presently disclosed and/or claimed inventive concept(s) are directed to formulation(s), kit(s), and/or rnethod(s) for protecting and/or suppressing and/or delaying the reoccurrence of cancer in a patient comprising administering effective amounts of biochemical prooxidants and/or antioxidant capacity reducing agents to a patient. The combination of biochemical prooxidant agents and/or antioxidant capacity reducing agents serve as additive, complementary, and/or synergistic cancer protection and/or suppressing agents through their generation of EMODs and the reduction of antioxidants. In addition, due to the lack of systemic toxicities of the majority of “whole” and “isolated” natural compounds and their potential to reduce toxic dosages and delay the development of drug resistances, makes biochemical prooxidant agents and/or antioxidant capacity reducing agents effective candidates for aiding in cancer management. Such biochemical prooxidant agents and/or antioxidant capacity reducing agents can be used in combination with traditional cancer therapies, including, but not limited to, radiation therapies, chemotherapies, and surgery.

Cancer cells exhibit increased intrinsic EMOD stress, due in part to the oncogenic stimulation, increased metabolic activity, and mitochondrial malfunction. The currently disclosed and/or claimed inventive concept(s) exploit these biochemical features to preferentially target and (suppress) eliminate cancer cells through EMOD-mediated mechanisms, The ability of malignant tumor cell populations to expand in number is not only a function of the rate of cell proliferation, but also by the rate of cell death. Apoptosis is a major source of cell death. Thus, agents that trigger apoptotic cancer cell death are promising candidates for cancer therapy; and cancer cells are vulnerable to high (as compared to a non-malignant cell) levels of EMODs that trigger the apoptotic cascade, while not killing normal cells.

EMODs (formerly referred to in the literature by inaccurate terms such as, activated oxygen, oxygen free radicals, reactive oxygen intermediates, ROS, and reactive oxygen metabolites) do not refer to the radicality, charge, or reactivity of the particular compound(s). Rather, the term “EMODs” indicates that the electron structure of ground state triplet di-radical oxygen has been altered and/or modified. Many EMODs have proven to be prooxidant apoptotic triggering agents, to which high levels can be achieved by either increasing EMOD-generating agents and/or by reducing antioxidant capacity(-ies).

EMODs include, but are not limited to, radical species of superoxide, hydroxyl, peroxyl, lipid peroxyl, alkoxyl, nitric oxide, and nitrogen dioxide. Additionally, while not free radicals themselves, the presently disclosed and/or claimed inventive concept(s) may also utilize, without limitation, hydrogen peroxide, ozone, singlet oxygen, hypochlorous acid, nitrous acid, peroxynitrite, dinitrogen trioxide, and lipid peroxides, as these compounds/molecules can easily lead to free radical creation in patients via physiologic and/or pathologic conditions. In accordance with the above, EMODs may comprise radical and non-radical species. In general, “free radicals” are defined as molecules or molecular fragments containing one or more unpaired electron(s) in its outer orbit, and this unpaired electron(s) is/are unstable and gives a significant degree of reactivity to the free radical as an electron acceptor.

EMODs can be generated by enzymatic reactions, including, but not limited to, enzymatic reactions involving nicotinamide adenine dinucleotide phosphate oxidases (“NADPH” or “NOXs”), xanthine oxidase, uncoupled endothelial nitric oxide synthase (“eNOS”), arachidonic acid, and metabolic enzymes, including, without limitation, cytochrome P450 enzymes, lipoxygenase, and cyclooxygenase. By way of example only, under physiological conditions, superoxide is generated by the one-electron reduction of molecular oxygen by NOXs in cellular cytosol. In addition, by way of example only, EMODs can be produced in cellular mitochondria by the electron transport chain (“ETC”) complexes I, II, and/or III, in which an electron leaked from the respiratory chain reacts with molecular oxygen. When generated in the cytosol, the cytosolic superoxide is converted to hydrogen peroxide by the catalytic enzyme superoxide dismutase (“SOD1”) within the cytoplasm and the mitochondrial intermembrane space. When generated in the mitochondria, superoxide is converted to hydrogen peroxide by superoxide dismutase II (“SOD2”) in the mitochondrial matrix. In addition, hydrogen peroxide can be produced as a byproduct of certain biochemical reactions, including, but not limited to, 6-oxidation in peroxisomes and protein oxidation within the endoplasmic reticulum.

The vulnerability of cancer cells to EMOD oxidative signals is a therapeutic target for the rational design of new antitumor and anticancer agents (or combinations thereof), the design(s) of which the presently disclosed and/or claimed inventive concept(s) are directed. In addition to their effects on cellular division, prooxidant cytotoxic anticancer agents induce oxidative stress by modulating the levels of EMODs, including, but not limited to, the levels of superoxide anion radicals, hydrogen peroxide, and hydroxyl radicals. Disproportional increases in intracellular EMODs have been shown to induce cancer cell cycle arrest, senescence, and apoptosis. This can be accomplished, for example, with cancer chemotherapy, depletion of cells from antioxidant proteins, and/or via the generation of EMODs by immune cells. Additionally, apoptosis is linked to an increase in mitochondrial oxidative stress that causes cytochrome C release, an irreversible event that leads to the activation of caspases and apoptosis. (see, e.g., Hu, et al. Role of Reactive Oxygen Species (ROS) in Apoptosis Induction. Apoptosis. 2000; 5(5):415-18). It has also been shown that superoxide generation through the Rac-1/NADPH oxidase pathway can also induce pro-apoptotic signaling. (see, e.g., Chung Y M, et al. Molecular Ordering of ROS Production, Mitochondrial Changes, and Caspase Activation During Sodium Salicylate-Induced Apoptosis. Free Radic, Biol. Med. 2003; 34(4):434-42). In human pancreatic cancer and glioma cells, activation of Erk 1/2 upon treatment with exogenous hydrogen peroxide triggers cellular apoptosis of such cells. (see, e.g, Zhang Y, et al. Overexpression of Copper Zinc Superoxide Dismutase Suppresses Human Glioma Cell Growth. Cancer Res. 2002; 62(4):1205-12). Of significance, in all of the above-recited signaling event, increased levels of EMODs turn on the apoptotic pathway thereby signaling cellular death.

EMODs have been shown to be involved in the pathogenesis of various diseases and conditions, including, without limitation, cancer and inflammation. While EMODs are in fact involved in malignant tumor cell proliferation, secretion, differentiation, and defense, increased levels of EMODs can, counter intuitively, induce cellular apoptosis and senescence. (see, e.g., Behrend L, et al. Reactive Oxygen Species in Oncogenic Transformation. Biochem. Soc. Trans. 2003 Dec.; 31(Pt. 6):1441-4).

As discussed herein, apoptosis is a normal physiological process that is required for the maintenance of cellular homeostasis. Apoptotic cell death is accompanied by a series of complex biochemical events and morphological cellular changes, including, without limitation, cell shrinkage, chromatin condensation, DNA fragmentation, membrane budding, and the appearance of membrane-associated apoptotic bodies. Targeted therapeutic strategies (including, chemo-suppression and chemo-protection) which utilize specifically tailored prooxidant agents and/or antioxidant capacity reducing agents that excessively increase intracellular EMODs to trigger the apoptotic signaling cascade are dependent on the tumor type, tumor stage, and type and concentration of endogenous and exogenous EMODs. One aspect of the presently disclosed and/or claimed inventive concept(s) is directed to formulation(s) that utilize compounds that increase intracellular EMODs to eliminate/kill cancer cells by decreasing these cells' antioxidant capacity(-ies), by acting at varying sites within the apoptotic triggering cascade. Such compounds may either inhibit antioxidant systems or inhibit specific signaling pathways that upregulate antioxidants in cancer cells. The resulting increase in EMODs subsequently induces cancer cell death via damaging functions to such cancer cells and/or via specific induction of apoptosis through death signaling pathways. One important advantage of the currently disclosed and/or claimed inventive concept(s) is that normal cells (i.e., non-malignant cells) are not significantly affected due to their lower basal EMODs levels and their relative independence of antioxidants. Accordingly, combination formulation(s) comprising prooxidant agents and/or antioxidant capacity reducing agents increase EMOD levels within malignant cells to thereby increase such EMOD levels above the threshold for apoptotic triggering toxicity to such malignant cells. Such combination(s), to which, in one aspect, the presently disclosed and/or claimed inventive concept(s) is/are directed, involve multi-component formulation(s) comprising prooxidant agents and/or antioxidant capacity reducing agents that combinatorically and synergistically target a diverse array of cancer cell death signaling pathways and pro-apoptotic mechanisms. Malignant tumor cells display a significant metabolic heterogeneity. As such, multiple points of anticancer attack are significantly advantageous.

In one embodiment of the presently disclosed and/or claimed inventive concept(s), the biochemical prooxidant agents forming chemo-protective and chemo-suppression formulation(s) comprise effective amounts of artemisinin (“ART”), superoxide dismutase (“SOD”), and glucose oxidase (“GOx”).

Artemisinin (“ART”) and/or its derivatives contain a sesquiterpene lactone that itself contains an endoperoxide bridge. This endoperoxide bridge, aside from being semi-unique to ART, is primarily responsible for ART's mechanism of action for its chemo-suppression and chemo-protective activities. ART's endoperoxide bridge is first activated via cleavage after reacting with heme and iron(II) oxide, which results in the generation of oxygen free radicals (EMODs) that, in turn, damage susceptible proteins. ART (and its semisynthetic derivatives) target patient cancer cells via intracellular prodrug activation with formation of EMODs triggered by redox-active iron ions. The importance of the endoperoxide bridge present in ART is further bolstered as previous studies have shown that ART cytotoxicity against various cancer cell lines is eliminated upon chemical replacement of the endoperoxide functional group by an ether bridge producing the redox-inactive deoxyartemisinin. The endoperoxide pharmacophore contained in ART (and its derivatives, including, without limitation, artesunate) imparts a unique chemical reactivity, and, unlike other organic peroxide compounds, ART displays drug-like stability. In addition, it is the endoperoxide pharmacophore of ART that imparts its potent anticancer targeting and activity. ART has been shown to inhibit malignant tumor growth and it induces cell cycle arrest, promote oncosis, inhibits malignant tumor angiogenesis, clonogenicity, and tissue invasion, and inhibits cancer metastasis in renal cell carcinoma (“RCC”) cells without significant toxicity. (see, e.g., Jeong da E, et al. Re-purposing the Anti-Malarial Drug Artesunate as a Novel Therapeutic Agent for Metastatic Renal Cell Carcinoma due to its Attenuation of Tumor Growth, Metastasis, and Angiogenesis. Oncotarget, 2015 Oct. 20; 6(32):33046-64). Accordingly, an effective amount of ART in the formulation(s), kit(s), and method(s) of the presently disclosed and/or claimed inventive concept(s) facilitates the anti-cancer properties of such formulation(s), kit(s), and method(s). An effective amount of ART comprises a range from about 50 milligrams to about 1,000 milligrams, and from about 100 milligrams to about 900 milligrams, and from about 150 milligrams to about 500 milligrams, and from about 200 milligrams to about 400 milligrams, and equal to or less than about 300 milligrams. In one non-limiting embodiment of the presently disclosed and/or claimed inventive concept(s), the effective amount of ART comprises a range from about 75 milligrams to about 300 milligrams. In another non-limiting embodiment, the effective amount of ART comprises a range from about 100 milligrams to about 1,000 milligrams.

Superoxide Dismutase(s) (“SOD” or “SODs”), including, without limitation, SOD1 and SOD2, is/are metalloenzymes which catalyze the dismutation of superoxide anion(s) to oxygen and hydrogen peroxide. SODs ubiquitously exist in eukaryotes and prokaryotes and utilize metal ions, including, but not limited to, copper (Cu²⁺), zinc (Zn²⁺), manganese (Mn²⁺), and/or iron (Fe²⁺) as cofactors. SODs play an important role in converting the superoxide radical into hydrogen peroxide. For example, and not by way of limitation, SOD2, also referred to as manganese SOD, contains an active site that has manganese as a transition metal for rapid electron exchange and is located in the mitochondria, a key organelle for producing EMODs. (see, e.g., Delira Robbins and Yunfeng Zhao. Manganese Superoxide Dismutase in Cancer Prevention. Antioxid. Redox Signal. 2014 Apr. 1; 20(10):1628-45), SOD, and in particular manganese SOD, is a prooxidant, which promotes the accumulation of hydrogen peroxide which leads to the activation of oncogenic pathways; and hydrogen peroxide has been shown to substantially eliminate colorectal xenografts via apoptosis. (see, e.g., Zhang H J, et al. Activation of Matrix Metalloproteinase-2 by Overexpression of Manganese Superoxide Dismutase in Human Breast Cancer MCF7 Cells Involves Reactive Oxygen Species. J. Biol. Chem. 2002; 277:20919-926 and Zhang Y, et al. Complete Elimination of Colorectal Tumor Xenograft by Combined Manganese Superoxide Dismutase with Tumor Necrosis Factor-Related Apoptosis-Inducing Ligand Gene Virotherapy. Cancer Res. 2006; 66:4291-98). In addition, recent evidence suggests that co-administration of SOD mirnetics with cytotoxic anticancer agents enhances prooxidant activity, resulting from SOD-derived hydrogen peroxide preferentially targeting rapidly dividing cancer cells with compromised peroxide metabolism and high level of endogenous oxidative stress. (see, e.g., Georg T. Wondrak. Redox-Directed Cancer Therapeutics: Molecular Mechanisms and Opportunities. Antioxid. Redox Signal. 2009 Dec.; 11(12):3013-69).

SOD, along with other components of the presently disclosed and/or claimed inventive concept(s), shows synergistic effects with other prooxidant agents. By way of example, and not by way of limitation, curcumin oil has been shown to preserve manganese SOD expression and activity and delayed cancer progression in vitro. (see, e.g., Schiffman S C, et al. The Association of Manganese Superoxide Dismutase Expression in Barret's Esophageal Progression with MnTBAP and Curcumin Oil Therapy. J. Surg. Res. 2012; 176:535-41).

Accordingly, an effective amount of SOD in the formulation(s), kit(s), and method(s) of the presently disclosed and/or claimed inventive concept(s) facilitates the anti-cancer properties of such formulation(s), kit(s), and method(s). An effective amount of SOD comprises a range from about 8 milligrams to about 1,000 milligrams, and from about 50 milligrams to about 900 milligrams, and from about 150 milligrams to about 500 milligrams, and from about 200 milligrams to about 400 milligrams, and equal to or less than about 300 milligrams. In one non-limiting embodiment of the presently disclosed and/or claimed inventive concept(s), the effective amount of SOD comprises an amount of from about 50 milligrams to about 500 milligrams,

Glucose oxidase (“GOx”) is a prooxidant enzyme that catalyzes the conversion of β-D-glucose and molecular oxygen to Dδ-glucono-δ-lactone and hydrogen peroxide. Hydrogen peroxide produced by GOx has been shown to be effective in preventing tumor growth in mice bearing not only ascites tumor(s) but also solid tumor(s). In addition, the chemo-suppression and chemo-preventive effects of GOx were enhanced by the combined administration of catalase inhibitors such as 3-aminotriazole, hydroxylamine, and sodium azide or the glutathione synthesis inhibitor buthionine-(S,R)-sulfoximine. (see, e.g., Higuchi, et al. Enhancement of the Antitumor Effect of Glucose Oxidase by Combined Administration of Hydrogen Peroxide Decomposition Inhibitors Together with an Oxygenated Fluorocarbon. Volume 82; Issue 8 (August 1991):942-49). Studies have shown a connection between the GOx enzyme and chemotherapeutic properties. Glucose oxidase serves at least a dual role in chemo-prevention and chemo-suppression: (1) GOx converts glucose, the primary source of energy and food for cancer cells, into hydrogen peroxide, thereby starving cancer cells of their primary food source; and (2) the hydrogen peroxide produced by the conversion of glucose by GOx allows for prooxidant EMOD attack on cancer cells. In certain instances, for instance, by way of example only, in order to function as a robust catalyst, GOx requires flavin adenine dinucleotide (“FAD”) as a cofactor, FAD is a common component in biological oxidation-reduction (redox reactions). Redox reactions involve a gain or loss of electrons from a molecule. In a GOx-catalyzed redox reaction, FAD functions as the initial electron acceptor and is reduced to FADH₂. FADH₂ is subsequently oxidized by molecular oxygen, which is subsequently reduced to hydrogen peroxide. Accordingly, an effective amount of GOx in the formulation(s), kit(s), and method(s) of the presently disclosed and/or claimed inventive concept(s) facilitates the anti-cancer properties of such formulation(s), kit(s), and method(s). An effective amount of GOx comprises a range from about 50 milligrams to about 1,000 milligrams, and from about 100 milligrams to about 900 milligrams, and from about 150 milligrams to about 500 milligrams, and from about 200 milligrams to about 400 milligrams, and equal to or less than about 300 milligrams. In one non-limiting embodiment of the presently disclosed and/or claimed inventive concept(s), the effective amount of GOx comprises a range from about 100 milligrams to about 1,000 milligrams.

In another non-limiting embodiment of the presently disclosed and/or claimed inventive concept(s), the biochemical prooxidant agents and/or antioxidant capacity reducing agents forming chemo-protective and chemo-suppression formulation(s) comprise effective amounts of artemisinin (“ART”), superoxide dismutase (“SOD”), and glucose oxidase (“GOx”), graviola, vitamin D3, and turmeric. ART, SOD, and GOx have previously been disclosed and discussed herein, and for purposes of brevity, will not be re-discussed in detail in the context of this non-limiting embodiment.

Graviola (also known as Annona muricata or soursop) and/or its isolates has been shown to function synergistically with other cancer therapeutic modalities to induce and trigger the apoptotic death pathway in cancer cells. Graviola is a member of the Annonaceae family and is a fruit tree with a long history of traditional health uses, including, but not limited to, cancer and parasitic infections. Numerous investigations have substantiated graviola's pro-health activities and effects, including, without limitation, graviola's benefits as an anticancer agent, anticonvulsant agent, anti-arthritic agent, antiparasitic agent, antimalarial agent, hepato-protective, and an antidiabetic agent. Phytochemical studies have revealed that annonaceous acetogenins are the major constituents of graviola and more than one hundred annonaceous acetogenins have been isolated from the leaves, barks, seeds, roots, and fruits of the graviola tree, Extensive phytochemical analysis on different parts of the graviola tree have shown the presence of various phyto-constituents and compounds, including, without limitation, alkaloids, megastigmanes, flavonol triglycerides, phenolic, cyclopeptides, and essential oils. However, as discussed hereinabove, graviola has been shown to be generally rich in annonaceous acetogenin compounds (“AGEs”). The biological activities or AGEs are primarily characterized with toxicity against cancer cells. (see, e.g., Moghadamtousi, et al. Annona muricata (Annonaceae): A review of its Traditional Uses, Isolated Acetogenins and Biological Activities. Int. J. Mol. Sci. 2015 Jul; 16(7):15625-58). addition, studies have shown that graviola isolates, including, but not limited to, ethyl acetate, water, and ethanolic extracts, have positive anti-cancer effects, including, without limitation, induction of apoptosis of cancer cells via the mitochondrial-mediated pathway. As a result of AGEs strong anticancer and antitumor properties, tablet formulations of the ethyl acetate-soluble fraction obtained from graviola's leaves have been produced as a cancer adjuvant therapy. (see, e.g., Elisya, et al. Tablet Formulation of the Ethyl Acetate Soluble Extract of Soursop (Annona muricata) Leaves. Asian J. Appl, Sci. 2014; 2:323-29). Accordingly, an effective amount of graviola in the formulation(s), kit(s), and method(s) of the presently disclosed and/or claimed inventive concept(s) facilitates the anti-cancer properties of such formulation(s), kit(s), and method(s). An effective amount of graviola comprises a range from about 50 milligrams to about 1,000 milligrams, and from about 100 milligrams to about 900 milligrams, and from about 150 milligrams to about 500 milligrams, and from about 200 milligrams to about 400 milligrams, and equal to or less than about 300 milligrams. In one non-limiting embodiment of the presently disclosed and/or claimed inventive concept(s), the effective amount of graviola comprises a range from about 100 milligrams to about 1,000 milligrams.

Vitamin D3 (cholecalciferol) and/or its derivatives and/or metabolites (such as, calcitriol) have been shown to synergistically function with other cancer therapeutic modalities to induce and/or trigger the apoptotic death pathway of cancer cells. Among 40,000 patients in the Health Professionals Study, an increase in the Vitamin D3 level of about 62.5 nanograms/milliliter was associated with a reduction in the risk of head neck, esophageal, and pancreatic cancers and acute leukemia by more than fifty percent. In addition, numerous preclinical studies indicate that exposing cancer cells (as well as vascular endothelial cells derived from tumors) to high concentrations of active metabolites of Vitamin D3 halts progression through cancer cell genesis, induces apoptosis, and slows and/or arrests growth of tumors in vivo. Vitamin D3 compounds, including, but not limited to, Vitamin D3 derivatives and metabolites, such as, by way of example only, calcitriol, inhibit the growth and even eliminate cancer cells in vitro and in vivo and have been shown to have a potentiating effects on therapeutic anticancer agents. Optimal potentiation is accomplished when calcitriol is administered before or simultaneous with chemotherapy treatment agents and the combination of calcitriol of cisplatin in squamous cell carcinoma cells in vitro also enhanced the apoptotic effects of calcitriol. (see, e.g., Trump et al. Vitamin D: Considerations in the Continued Development as an Agent for Cancer Prevention and Therapy. Cancer. J. 2010. Jan.-Feb.; 16(1):1-9). In model systems of murine squamous cell carcinoma and human carcinomas arising in the prostate, lung, ovary, breast, bladder, and pancreas (as well as neuroblastornas), calcitriol and its derivatives have been shown to exhibit substantial anticancer effects. (see, id.). Calcitriol and/or its derivatives act via the vitamin D receptor (“VDR”) to regulate the differentiation, proliferation, apoptosis, and angiogenesis of cancer cells. New research suggests that calcitriol has significant protective effects against the development of cancer due to Vitamin D3's role as a nuclear transcription factor that regulates cell growth, differentiation, apoptosis, and a wide range of cellular mechanisms central to cancer development. (see, e.g., Ingraham, et al. Molecular Basis of the Potential of Vitamin D to Prevent Cancer. Curr. Med. Res. Opin. 2008. Jan.; 24(1):139-49). Incubation of keratinocytes with calcitriol or its low calcemic analogues, such as 20(OH)D3, 21(OH)pD, or calcipotriol, sensitized these cells to EMODs resulting in more potent inhibition of keratinocyte proliferation by hydrogen peroxide in the presence of Vitamin D3 compounds. In addition, studies have shown calcitriol and its analogues stimulate the expression of SOD1 and catalase (“CAT”) genes, but not SODII, indicating a possible role of mitochondria in EMOD-modulated cell death. (see, e.g., Piotrowska, et al. Vitamin D Derivatives Enhance Cytotoxic Effects of H2O2 or Cisplatin on Human Keratinocytes. Steroids. 2016 Jun.; 110:49-61). Accordingly, an effective amount of vitamin D3 in the formulation(s), kit(s), and method(s) of the presently disclosed and/or claimed inventive concept(s) facilitates the anti-cancer properties of such formulation(s), kit(s), and rnethod(s). An effective amount of vitamin D3 comprises a range from about 50 milligrams to about 2,000 milligrams, and from about 100 milligrams to about 1,900 milligrams, and from about 150 milligrams to about 1,500 milligrams, and from about 200 milligrams to about 1,000 milligrams, and equal to or less than about 1,000 milligrams. In one non-limiting embodiment of the presently disclosed and/or claimed inventive concept(s), the effective amount of vitamin D3 comprises a range from about 100 milligrams to about 2,000 milligrams.

Turmeric (curcumin or Curcuma longa) has been shown to work synergistically with other cancer therapeutic modalities to induce and/or trigger the apoptotic death pathway of cancer cells. Certain types of cancers are more prevalent in some countries than in others, suggesting that lifestyle, including, but not limited to, a patient's diet, plays an important role in cancer genesis. Among potential dietary contributors to this disparity is turmeric, a spice that is frequently consumed by persons from southeast Asia, a continent with a relatively low incident of most cancers. Powder of turmeric is routinely arid extensively used in Ayurveda, Unani, and Siddha medicine as a remedy for various ailments and diseases. This powder (curcumin or diferyloylrnethane) is a yellow-colored polyphenol identified as 1,6-heptadiene-3,5-dione-1,7-bis(4-hydroxy-3-methoxyphenyl)-(1E,6E). In addition, turmeric contains minor fractions such as dernethoxycurcurnin (curcurnin II), bisdemethoxycurcumin (curcumin III), and cyclocurcumin. Curcumin has a diverse range of molecular targets, supporting the concept that curcumin acts upon numerous biochemical and molecular cascades. (see, e.g., Ravindran, et al. Curcumin and Cancer Cells: How Many Ways can Curry Kill Tumor Cells Selectively? AAPSJ 2009. Sept; 11(3):495-510). Curcumin has been shown to suppress multiple signaling pathways and inhibit cell proliferation, invasion, metastasis, and angiogenesis. The chemo-preventive characteristics of curcumin may be due to its ability to induce apoptosis via several pathways. The pro-apoptotic activity of curcumin has been reported to be inhibited by SOD and N-acetyl cysteine in leukemia cells, suggesting the involvement of superoxide radicals. (see, e.g., Kuo, et al. Curcumin, an Antioxidant and Anti-Tumor Promoter, induces Apoptosis in Human Leukemia Cells. Biochim, Biophys. Acta. 1996. Nov. 15; 1317(2):95-100). The antioxidants N-acetyl-L-cysteine, L-ascorbic acid, alpha-tocopherol, catalase, and SOD all effectively prevent curcumin-induced apoptosis. This suggests that curcurnin-induced cell death is mediated by EMODs, which is further exemplified by mannitol (a hydroxyl radical quencher) had no effect on the pro-apoptotic activity of curcumin. The totality of the evidence suggests that the pro-apoptotic effects of curcumin are mediated through a prooxidant pathway, potentially via the activation of mitochondrial enzymes that lead to production of EMODs. (see, Sandur, et al. Role of Prooxidants and Antioxidants in the Anti-inflammatory and Apoptotic Effects of Curcumin (Diferuloylmethane), Free Radic. Biol. Med. 2007. Aug. 15; 43(4):568-80). Accordingly, an effective amount of turmeric (curcumin) in the formulation(s), kit(s), and method(s) of the presently disclosed and/or claimed inventive concept(s) facilitates the anti-cancer properties of such formulation(s), kit(s), and method(s). An effective amount of turmeric (curcumin) comprises a range from about 50 milligrams to about 1,000 milligrams, and from about 100 milligrams to about 900 milligrams, and from about 150 milligrams to about 500 milligrams, and from about 200 milligrams to about 400 milligrams, and equal to or less than about 300 milligrams. In one non-limiting embodiment of the presently disclosed and/or claimed inventive concept(s), the effective amount of turmeric (curcumin) comprises a range from about 100 milligrams to about 1,000 milligrams.

In another non-limiting embodiment of the presently disclosed and/or claimed inventive concept(s), the biochemical prooxidant agents and/or antioxidant capacity reducing agents forming chemo-protective and chemo-suppression formulation(s) comprise effective amounts of artemisinin (“ART”), superoxide dismutase (“SOD”), and glucose oxidase (“GOx”), vitamin D3, turmeric, sulforaphane (“SFN”), cinnamaldehyde, and garlic extract. ART, SOD, and GOx, vitamin D3, and turmeric have previously been disclosed and discussed herein, and for purposes of brevity, will not be re-discussed in detail in the context of this non-limiting embodiment.

Sulforaphane (“SFN”) is a naturally occurring organosulphur compound that is created as a result of an enzymatic process when cruciferous vegetable(including, but not limited to, broccoli, cauliflower, and kale) cells are broken. The enzyme myrosinase converts glucoraphanin, a glucosinolate, into SFN upon damage to the plant and the breaking of the cell wail (such as via chewing). SFN is quickly and easily absorbed by a patient's body when ingested. SFN has been extensively studied due to its apparent health-promoting and limited toxicity in normal tissue and cells. SFN mediates a number of anticancer pathways, including, without limitation, apoptosis, induction of cell cycle arrest, and inhibition of nuclear factor kappa-light-chain-enhancer of activated B cells (“NFκB”). Studies have reported an inverse association with an increase in cruciferous vegetable(s) consumption and a patient's cancer risk, including malignancies of the breast, lung, prostate, pancreas, and colon. (see, e.g., Tortorella, et al. Dietary Sulforphane in Cancer Chemoprevention: The Role of Epigenetic Regulation and HDAC Inhibition. Antioxid. Redox. Signal. 2015. Jun. 1; 22(16):1382-1424). The initiating signal of SFN-mediated apoptosis is the formation of EMODs and the disruption of mitochondrial membrane potential, leading to cytosolic release of cytochrome C via both death-receptor and mitochondrial caspase cascade pathways. (see, e.g., Singh, et al. Sulforphane-Induced Cell Death in Human Prostate Cancer Cells is Initiated by Reactive Oxygen Species. J. Biol. Chem. 2005. May 20; 280(20):19911-24). In addition, SFN is capable of inducing apoptosis through the activation of EMOD-dependent caspase-3 in multiple tumor necrosis factor-α-resistant leukemia cell lines. (see, e.g., Moon, et al. Sulforphane Suppresses TNF-Alpha-Mediated Activation of NF-kappaB and Induces Apoptosis through Activation of Reactive Oxygen Species-Dependent Caspase-3. Cancer Lett. 2009. Feb. 8; 274(1):132-42). SFN has the ability to modulate both extrinsic and intrinsic apoptotic pathways via the production of EMODs and regulation of gene expression. The initial signal for SFN-induced apoptosis is derived from EMODs. It has been shown that exposure of prostate cancer-3 cells to growth-suppressive concentrations of SFN resulted in EMOD generation, which was accompanied by disruption of mitochondrial membrane potential, cytosolic release of cytochrome C, and apoptosis. (see, e.g., Singh, et al. referred to hereinabove). The chemo-protective properties of SFN and its capacity to be selectively toxic to malignant cells and impart these effects through a plurality of mechanisms, elucidate the impact that SFN can have as an anticancer, EMOD-generating agent. Accordingly, an effective amount of SFN in the formulation(s), kit(s), and method(s) of the presently disclosed and/or claimed inventive concept(s) facilitates the anti-cancer properties of such formulation(s), kit(s), and method(s). An effective amount of SFN comprises a range from about 50 milligrams to about 1,000 milligrams, and from about 100 milligrams to about 900 milligrams, and from about 150 milligrams to about 500 milligrams, and from about 200 milligrams to about 400 milligrams, and equal to or less than about 300 milligrams. In one non-limiting embodiment of the presently disclosed and/or claimed inventive concept(s), the effective amount of SFN comprises a range from about 100 milligrams to about 1,000 milligrams.

Cinnamaidehyde is an active compound isolated from the stem bark of Cinnamomum cassia, a traditional Chinese medicinal herb, which has been shown to inhibit tumor cell proliferation. Cinnamaldehyde is a potent inducer of apoptosis and it transduces the apoptotic signal via EMOD generation, thereby inducing mitochondrial permeability transition and cytochrome C release in the cytosol. (see, e.g., Ka, et al. Cinnamaldehyde induces Apoptosis by ROS-Mediated Mitochondrial Permeability Transition in Human Promyelocytic Leukemia. Cancer Lett. Jul. 10, 2003. Vol. 196, Issue 2, Pages 143-52). 2′-hydroxycinnamaldehyde (“HCA”) has been shown to have inhibitory effects on farnesyl protein transferase in vitro, angiogenesis, and tumor cell growth. HCA and/or 2′-benzoyl-oxycinnamaldehyde (“BCA”), a derivative of HCA, have been shown even at low concentrations (about 10 μM) to inhibit growth and induce apoptosis of tumor cells. Markers of apoptosis, including, without limitation, degradations of chromosomal DNA and poly(ADP-ribose) polymerase and activation of caspase-3 were detected after HCA and/or BCA treatment. In addition, BCA-induced apoptosis was blocked by pretreatment of the tumor cells with antioxidants, glutathione, or N-acetylcysteine, and, the degradative effects spawned by BCA-induced activation were eliminated by the pre-treatment of the cells with antioxidants. These results suggest that EMODs are a major regulator of BCA-induced apoptosis, and that cinnamaldehyde and its derivatives are promising candidates for cancer therapy(-ies). (see, e.g., Han, et al. 2′-Benzoyloxycinnamaldehyde induces Apoptosis in Human Carcinoma Via Reactive Oxygen Species. J. Biol. Chem. 2004. Feb. 20; 279:6911-20). Accordingly, an effective amount of cinnamaldehyde in the formulation(s), kit(s), and rnethod(s) of the presently disclosed and/or claimed inventive concept(s) facilitates the anti-cancer properties of such formulation(s), kit(s), and method(s). An effective amount of cinnamaldehyde comprises a range from about 100 milligrams to about 2,000 milligrams, and from about 200 milligrams to about 1,900 milligrams, and from about 300 milligrams to about 1,500 milligrams, and from about 500 milligrams to about 1,000 milligrams, and equal to or less than about 1,000 milligrams. In one non-limiting embodiment of the presently disclosed and/or claimed inventive concept(s), the effective amount of cinnamaldehyde comprises a range from about 500 milligrams to about 2,000 milligrams.

Garlic (Allium sativum) is among the oldest of all cultivated plants and has been used as a medicinal agent for millennia due to its antimicrobial, antithrombotic, hypo-lipidemic, anti-arthritic, hypoglycemic, and antitumor activities and properties, A number of studies have demonstrated the chemo-preventive activity of garlic by using different garlic preparations, including, but not limited to, fresh garlic extract, aged garlic, garlic oil, and a number of organosulfur compounds derived from garlic. Garlic's chemo-preventive activity has been attributed to the presence of the organosulfur compounds, including, without limitation, S-allylcysteine, which have been found to retard the growth of chemically-induced and transplantable tumors in several animal models. The anti-neoplastic activity of garlic has been studied in mice injected with cancer cells pretreated with garlic extract. No deaths occurred in this treatment group for up to six months, while mice injected with untreated cancer cells die within about sixteen (16) days. It is believed that the reaction of allicin with sulfhydryl groups (the concentration of which greatly increases in rapidly-dividing cells, such as cancer cells) contributes to this inhibitory effect. Studies directed to the investigation of the effects of crude garlic extract (“CGE”) on the proliferation of human breast, prostate, hepatic, and colon cancer cells and mouse macrophageal cells have been published. It was found that cells, after overnight incubation, treated with 0.125, 0.25, 0.5, or 1 μg/mL of CGE and, after 72 hours of additional incubation, there was 80-90% inhibition of cell proliferation of Hep-G2, MCF-7, TIB-71, and PC-3 cells, but only 40-55% inhibition of cell proliferation of Caco-2 cells. However, in a co-culture study of Caco-2 and TIB-71 cells, there was 90% inhibition of cell proliferation. In addition, CGE also induced cell cycle arrest and had a fourfold increase in caspase activity (indicating apoptosis) in PC-3 cells when treated with a dose CGE having a concentration of 0.5 or 1 μg/mL. This study clearly highlights that the lipid bioactive compounds in CGE are promising anticancer agents. (see, e.g., Bagul, et al. Crude Garlic Extract Inhibits Cell Proliferation and Induces Cell Cycle Arrest and Apoptosis of Cancer Cell in Vitro. J. Med. Food. 2015. July; 18(7):731-37).

Garlic compounds have recently received increased attention due their chemo-preventive properties and anticancer activities. The effects of hexane extracts of garlic cloves (“HEGCs”) on EMODs production and the association of these effects with apoptotic cell death have been studied using in vitro Hep3B human hepatocarcinoma cell lines. The results demonstrated that HEGCs mediate EMOD production and that this mediation is followed by collapse of mitochondrial membrane potential (“MMP”), the down regulation of anti-apoptotic Bcl-2 and Bcl-xL, and the activation of caspase-3 and caspase-9. HEGCs also promoted the activation of caspase-8 and the down regulation of Bid, a BH3-only pro-apoptotic member of the Bcl-2. Moreover, N-acetyl-L-cysteine, a widely used EMOD scavenger, effectively blocked HEGC-induced apoptotic effects via the inhibition of EMOD production and MMP collapse. These observations clearly indicate that HEGC-induced EMODs are key mediators of MMP collapse, which leads to the induction of apoptosis followed by caspase activation. (see, e.g., Kim, et al. Hexane Extracts of Garlic Cloves induce Apoptosis through the Generation of Reactive Oxygen Species in Hep3B Human Hepatocarcinorna Cells. Oncology Reports. Aug. 23, 2012. Pages: 1757-63). Accordingly, an effective amount of garlic extract in the formulation(s), kit(s), and methods) of the presently disclosed and/or claimed inventive concept(s) facilitates the anti-cancer properties of such formulation(s), kit(s), and method(s). An effective amount of garlic extract comprises a range from about 100 milligrams to about 10,000 milligrams, and from about 200 milligrams to about 19,000 milligrams, and from about 300 milligrams to about 18,000 milligrams, and from about 400 milligrams to about 17,000 milligrams, and from about 500 milligrams to about 16,000 milligrams, and from about 600 milligrams to about 15,000 milligrams, and from about 700 milligrams to about 14,000 milligrams, and from about 800 milligrams to about 13,000 milligrams, and from about 900 milligrams to about 12,000 milligrams, and from about 1,000 milligrams to about 11,000 milligrams, and equal to or less than about 10,000 milligrams. In one non-limiting embodiment of the presently disclosed and/or claimed inventive concept(s), the effective amount of garlic extract comprises a range from about 1,000 milligrams to about 10,000 milligrams.

In another non-limiting embodiment of the presently disclosed and/or claimed inventive concept(s), the biochemical prooxidant agents and/or antioxidant capacity reducing agents forming cherno-protective and chemo-suppression formulation(s) comprise effective amounts of artemisinin (“ART”), superoxide dismutase (“SOD”), and glucose oxidase (“GOx”), vitamin D3, turmeric, and alpha lipoic acid (“ALA”). ART, SOD, and GOx, vitamin D3, and turmeric have previously been disclosed and discussed herein, and for purposes of brevity, will not be re-discussed in detail in the context of this non-limiting embodiment.

Alpha lipoic acid (“ALA”) (also known as thioctic acid or 5-(1,2-dithiolan-3-yl)pentanoic acid) is a naturally occurring antioxidant synthesized in small amount by plants and animals, including humans, which functions as an essential co-factor for several mitochondrial multi-enzyme complexes involved in energy metabolism. ALA has been shown to regenerate other major antioxidants and protect the body from oxidative stress. Research has also shown that ALA has the ability to outperform chemotherapy in its ability to reduce cancer cell formation, with little to no side effects. ALA is found in food and many alternative health therapies to aid patients suffering from diabetes, neurodegenerative conditions, autoimmune diseases, cancer, and heart disease. In cells, ALA is reduced to its active form, dihydrolipoic acid, which actively scavenges various EMODs and regenerates other endogenous antioxidants. Considerable attention has recently been focused on ALA's potential anticancer effects as ALA is capable of inducing cell cycle arrest, suppress proliferation, and induce apoptosis in different cancer cell lines. The antitumor activity of ALA observed in MCF-7 breast cancer cell line depicts the importance of the redox state in regulating cancer initiation and progression. (see, e.g., Dozio, et al. The Natural Antioxidant Alpha-Lipoic Acid Induces p27Kip1-Dependent Call Cycle Arrest and Apoptosis in MCF-7 Human Breast Cancer Cells. Euro. J. of Pharma. 2010. 641:29-34). It has been reported that ALA induces EMOD generation and a concomitant increase in apoptosis of human lung epithelial cancer H460 cells. Inhibition of EMOD generation by EMOD scavengers or by overexpression of antioxidant enzymes giutathione peroxidase and SOD effectively inhibited ALA-induced apoptosis. This finding indicates the important role of EMODs, including, without limitation, hydrogen peroxide and superoxide anion, in the apoptotic process. Apoptosis induced by ALA has been shown to be mediated through the mitochondrial death pathway, which requires caspase-9 activation. In addition, inhibition of caspase activity by the pan-caspase inhibitor (z-VAD-FMK) or caspase-9-specific inhibitor (z-LEND-FMK) completely inhibited the apoptotic effect of ALA. (see, e.g., Moungjaroen, et al. Reactive Oxygen Species Mediate Caspase Activation and Apoptosis induced by Lipoic Acid in Human Lung Endothelial Cancer Cells through Bcl-2 Down-Regulation. J. of Pharma. And Exp. Thera. 2006. Dec.; 319(3):1062-69). Accordingly, an effective amount of ALA in the formulation(s), kit(s), and method(s) of the presently disclosed and/or claimed inventive concept(s) facilitates the anti-cancer properties of such formulation(s), kit(s), and method(s). An effective amount of ALA comprises a range from about 100 milligrams to about 1,000 milligrams, and from about 200 milligrams to about 900 milligrams, and from about 300 milligrams to about 800 milligrams, and from about 400 milligrams to about 700 milligrams, and equal to or less than about 600 milligrams. In one non-limiting embodiment of the presently disclosed and/or claimed inventive concept(s), the effective amount of cinnamaldehyde comprises a range from about 300 milligrams to about 600 milligrams.

While certain compounds have been discussed herein with respect to the formulation(s), kit(s), and/or method(s) of the presently disclosed and/or claimed inventive concept(s), a person having reasonable skill in the art should appreciate that such inventive concept(s) are not limited to these specific compounds. Accordingly, the formulation(s), kit(s), and/or rnethod(s) of the presently disclosed and/or claimed inventive concept(s) may be fully realized with a number of different prooxidant agents and/or antioxidant capacity reducing agents, including, but not limited to: abrin, ajoene, allicin, benzyl isothiocyanate, diallyl disulfide, dimethyl disulfide, jasmonic acid, linoleic acid, linolenic acid, L-mimosine, melatonin, methyl jasmonate, phenylethylisothiocyanate, sorbitol, 2′-hydroxycinnamaldehyde, 3,7,4′-trihydroxyflavone, 4′-hydroxycinnamaidehyde, 4-hydroxycinnamic acid, 6-dehydrogingerdione, 6-gingerol, 6-shogaol, 8-shogaol, acacetin, aesculetin, aloe--emodin, apigenin, baicalein, baicalin benzaldehyde, betuletol 3-methyl ether, butein, caffeic acid, cajanol, catechin catechol, chebulinic acid, chlorogenic acid, chrysin, chrysoeriol, chrysophanol, cyaniding, cyanidin 3-glucoside, cyanidin-3-rutinoside, daidzein, dantron, daphnetin, delphinidin, delphinidin 3-sambubioside, diospyrin, ellagic acid, emodin, epicatechin, epicatechin-gallate, epigallocatechin, epigallocatechin-3-gallate, eriodictyol, esculetin (aesculetin), eugenol, eupafolin, ferulic acid, fisetin, flavokawain B, fraxetin, gallic acid, gambogic acid, genistein, gentiacaulein, gentiakochianin, guttiferone-A, hesperetin, hydroxytyrosol, icarlin, isoeugenol, isoliquiritigenin, juglone, kaempferol, liquiritigenin, luteolin, rnalvidin, rnalvidin 3-glucoside, methyl gallate, morin, rnyricetin, naringenin, nordihydroguaiaretic acid, norwogonin, pelargonidin, pelargonidin 3-glucoside, pentagalloyl glucose, peonidin, peonidin 3-glucoside, phloretin, plurnbagin, procyanidin, protoapigenone, psoralen, pterostilbene, quercetin, resveratrol, rhein, rosmarinic acid, rottlerin, rutin, salicylic acid, shikonin, sinapic acid, sophoranone, tannic acid, taxifolin, tricetin, usnic acid, vanillin, wogonin, xanthohumol, xanthotoxin, 18b-glycyrrhetinic acid, andrographolide, asiatic acid, astilbotriterpenic acid, betulinic acid, bixin, bufalin ,cannabidiol, costunolide, cucurbitacin B, dioscin, diosgenin, erythrodiol, farnesol, ginkgolide B, ginsenoside RH-2, glaucocalyxin A, guggulsterone, gypenosides, helenalin, linalool, lupeol, lycopene, oleandrin, oleanolic acid, oleuropein, oridonin, ouabain, ovatodiolide, taxol, parthenolide, perillyl alcohol, polygodial, pristimerin, protopanaxadiol, sarsasapogenin, tetrahydrocannabinol, thymol, triptolide, ursolic acid, uvaol, withaferin, a-hederin, a-humulene, b-arnyrin, b-carotene, b-escin (aescin), atractyloside, b-sitosterol, vernolepin, 6-methoxydihydrosanguinarine, berberine, boldine, caffeine, carnptothecin, cepharanthine, chelerythrine, ellipticine, hornoharringtonine, indole acetic acid, indole-3-carbinol, lycopodine, morphine, oxymatrine, pancratistatin, piperine, sampangine, sanguinarine, tetrandrine, tornatine, vinblastine, vincristine, 4-acetyl-12,13-epoxyl-9-trichothecene-3, 15-diol, aclarubicin, actinomycin-D, aplidin, arachidonic acid, ascididernin, bleomycin, boningrnycin, butenolide, capsaicin, chenodeoxycholic acid, cholic acid, C-phycocyanin, cribrostatin 6, daunomycin anthracycline, deoxycholic acid, deoxynivalenol, docosahexaenoic acid, doxorubicin, eicosapentaenoic acid, F-2 mycotoxin, fucoxanthin, isoobtusilactone A, kotomolide A, mitomycin C, neocarzinostatin, norharman, ochratoxin A, patulin, putrescine-1,4-dicinnamide, secotenuifolide, T-2 mycotoxin, ursodeoxycholic acid, vitamin A, vitamin C, vitamin D2, vitamin K2, and/or vitamin K3.

The presently disclosed and/or claimed inventive concept(s) further embody method(s) of use and/or administration of the prooxidant agent(s) and/or antioxidant capacity reducing agents to a patient suffering from cancerous and/or tumorous conditions to thereby trigger apoptosis of such cancerous and/or tumorous cells. The method(s) comprise administering a chemo-suppression and/or chemo-preventive composition to a patient, followed by subsequent triggering of apoptosis in and to the cancerous and/or tumorous cells. As discussed herein, the chemo-suppression and/or chemo-preventive composition can be: (1) a single prooxidant agent or antioxidant capacity reducing agent disclosed herein; or (2) a plurality combination of prooxidant agents or antioxidant capacity reducing agents disclosed herein. The chemo-suppression and/or chemo-preventive composition may be administered to a patient via any carrier customary in the art, including, but not limited to, soft-gel capsules, hard-shell capsules, tablets, time-release capsules, cocktails, liquids (including drops), and rectal administration, including, without limitation, rectal suppositories. Once administered to a patient, the chemo-suppression and/or chemo-preventive composition comprising prooxidant agents and/or antioxidant capacity reducing agents are metabolized within the patient to produce EMODs that trigger the apoptotic death pathway in cancerous and/or tumorous cells (while minimally, if at all, associating with normal, healthy cells) to thereby kill the cancerous and/or tumorous cells.

NON-LIMITING EXAMPLES OF THE INVENTIVE CONCEPT(S)

A chemo-suppression and chemo-protection composition, comprising: an effective amount of a plurality of electronically modified oxygen derivative generating compounds, wherein the electronically modified oxygen derivative generating compounds are metabolized into a plurality of electronically modified oxygen derivatives which thereby induce a cellular apoptotic cascade within a population of tumorous or cancerous cells.

The composition, wherein the electronically modified oxygen derivative generating compounds are selected from the group consisting artemisinin, superoxide dismutase, and glucose oxidase.

The composition, further comprising electronically modified oxygen generating compounds selected from the group consisting of graviola, vitamin D3, turmeric, sulforaphane, cinnamaldehyde, garlic extract, and alpha lipoic acid.

The composition, wherein the artemisinin comprises from about 100 milligrams to about 1,000 milligrams.

The composition, wherein the superoxide dismutase comprises from about 50 milligrams to about 500 milligrams.

The composition, wherein the glucose oxidase comprises from about 100 milligrams to about 1,000 milligrams.

The composition, wherein the graviola comprises from about 100 milligrams to about 1,000 milligrams.

The composition, wherein the vitamin D3 comprises from about 100 milligrams to about 2,000 milligrams.

The composition, wherein the turmeric comprises from about 100 milligrams to about 1,000 milligrams.

The composition, wherein the sulforaphane comprises from about 100 milligrams to about 1,000 milligrams.

The composition, wherein the cinnamaldehyde comprises from 500 milligrams to about 2,000 milligrams.

The composition, wherein the garlic extract comprises from about 1,000 milligrams to 10,000 milligrams.

The composition, wherein the alpha lipoic acid comprises from about 300 milligrams to about 600 milligrams.

The composition, wherein the electronically modified oxygen derivatives are selected from the group consisting of peroxides, superoxides, hydroxyl radicals, singlet oxygen, superoxide anions, and alkoxides.

The composition, wherein the peroxide is hydrogen peroxide.

A method of inducing apoptosis in a population of cancer cells, the method comprising the steps of: administering a cherno-suppression composition comprising an effective amount of a plurality of electronically modified oxygen derivative generating compounds; and triggering at least one apoptotic enzyme by metabolizing the plurality of electronically modified oxygen derivative generating compounds, thereby generating electronically modified oxygen derivatives which thereby induce a cellular apoptotic cascade within a population of cancer cells.

The method, wherein the triggering of apoptotic enzymes occurs within mitochondria of the population of cancer cells.

The method, wherein the at least one apoptotic enzyme is caspase-9.

Thus, in accordance with the presently disclosed and claimed inventive concept(s), there have been provided devices, kits, and methods for detecting at least one analyte present in a patient's low-volume liquid test sample. As described herein, the presently disclosed and claimed inventive concept(s) relate to embodiments of formulation(s) of prooxidant agents and/or antioxidant capacity reducing agents that, when metabolized, generate EMODs that trigger the apoptotic cascade in tumorous and/or cancer cells, as well as kits and methods of use related thereto. Such presently disclosed and/or claimed inventive concept(s) fully satisfy the objectives and advantages set forth hereinabove. Although the presently disclosed and claimed inventive concept(s) has been described in conjunction with the specific drawings, experimentation, results and language set forth hereinabove, it is evident that many alternatives, modifications, and variations will be apparent to those skilled in the art. Accordingly, it is intended to embrace all such alternatives, modifications and variations that fall within the spirit and broad scope of the presently disclosed and claimed inventive concept(s).

### REFERENCES

The following references, to the extent that they provide exemplary procedural and/or other details supplementary to those set forth herein, are specifically incorporated herein by reference. In addition, the following is not intended to be an Information Disclosure Statement; rather, an Information Disclosure Statement in accordance with the provisions of 37 CFR § 1.97 will be submitted separately.

Hu, et al. Role of Reactive Oxygen Species (ROS) in Apoptosis Induction. Apoptosis. 2000; 5(5):415-18.

Chung Y M, et al. Molecular Ordering of ROS Production, Mitochondrial Changes, and Caspase Activation During Sodium Salicylate-Induced Apoptosis. Free Radic. Biol. Med. 2003; 34(4):434-42.

Zhang Y, et al. Overexpression of Copper Zinc Superoxide Dismutase Suppresses Human Glioma Cell Growth. Cancer Res. 2002; 62(4):1205-12.

Behrend L, et al. Reactive Oxygen Species in Oncogenic Transformation. Biochem. Soc. Trans. 2003 Dec.; 31(Pt. 6):1441-4.

Jeong da F, et al. Re-purposing the Anti-Malarial Drug Artesunate as a Novel Therapeutic Agent for Metastatic Renal Cell Carcinoma due to its Attenuation of Tumor Growth, Metastasis, and Angiogenesis. Oncotarget. 2015 Oct. 20; 6(32):33046-64.

Delira Robbins and Yunfeng Zhao. Manganese Superoxide Dismutase in Cancer Prevention. Antioxid, Redox Signal. 2014 Apr. 1; 20(10):1628-45.

Zhang H J, et al. Activation of Matrix Metalloproteinase-2 by Overexpression of Manganese Superoxide Dismutase in Human Breast Cancer MCF7 Cells Involves Reactive Oxygen Species. J. Biol. Chem. 2002; 277:20919-926.

Zhang Y, et al. Complete Elimination of Colorectal Tumor Xenograft by Combined Manganese Superoxide Dismutase with Tumor Necrosis Factor-Related Apoptosis-inducing Ligand Gene Virotherapy. Cancer Res. 2006; 66:4291-98.

Georg T. Wondrak, Redox-Directed Cancer Therapeutics: Molecular Mechanisms and Opportunities. Antioxid. Redox Signal. 2009 Dec.; 11(12)3013-69.

Schiffman S C, et al. The Association of Manganese Superoxide Dismutase Expression in Barret's Esophageal Progression with MnTBAP and Curcumin Oil Therapy. J. Surg. Res. 2012; 176:535-41.

Higuchi, et al. Enhancement of the Antitumor Effect of Glucose Oxidase by Combined Administration of Hydrogen Peroxide Decomposition Inhibitors Together with an Oxygenated Fluorocarbon. Volume 82; Issue 8 (August 1991):942-49.

Moghadamtousi, et al. Annona muricata (Annonaceae): A review of its Traditional Uses, Isolated Acetogenins and Biological Activities. Int. J. Mol. Sci. 2015 Jul; 16(7):15625-58.

Elisya, et al. Tablet Formulation of the Ethyl Acetate Soluble Extract of Soursop (Annona muricata) Leaves. Asian J. Appl. Sci. 2014; 2:323-29.

Trump et al. Vitamin D: Considerations in the Continued Development as an Agent for Cancer Prevention and Therapy. Cancer. J. 2010. Jan.-Feb.; 16(1):1-9.

Ingraham, et al. Molecular Basis of the Potential of Vitamin D to Prevent Cancer. Curr. Med. Res. Opin. 2008. Jan.; 24(1)139-49.

Piotrowska, et al. Vitamin D Derivatives Enhance Cytotoxic Effects of H2O2 or Cisplatin on Human Keratinocytes. Steroids. 2016 Jun.; 110:49-61.

Ravindran, et al. Curcumin and Cancer Cells: How Many Ways can Curry Kill Tumor Cells Selectively? AAPS J. 2009. Sep.; 11(3):495-510.

Kuo, et al. Curcumin, an Antioxidant and Anti-Tumor Promoter, Induces Apoptosis in Human Leukemia Cells. Biochim. Biophys, Acta. 1996. Nov 15; 13:17(2):95-100.

Sandur, et al. Role of Prooxidants and Antioxidants in the Anti-Inflammatory and Apoptotic Effects of Curcumin (Diferuloylmethane). Free Radic. Biol. Med. 2007. Aug. 15; 43(4):568-80.

Tortorella, et al. Dietary Sulforphane in Cancer Chemoprevention: The Role of Epigenetic Regulation and HDAC Inhibition. Antioxid. Redox. Signal. 2015. Jun. 1; 22(16):1382-1424.

Singh, et al. Sulforphane-Induced Cell Death in Human Prostate Cancer Cells is initiated by Reactive Oxygen Species. J. Biol. Chem. 2005. May 20; 280(20):19911-24.

Moon, et al. Sulforphane Suppresses TNF-Alpha-Mediated Activation of NF-kappaB and Induces Apoptosis through Activation of Reactive Oxygen Species-Dependent Caspcise-3. Cancer Lett, 2009. Feb. 8; 274(4132-42.

Ka, et al. Cinnamaldehyde induces Apoptosis by ROS-Mediated Mitochondrial Permeability Transition in Human Promyelocytic Leukemia. Cancer Lett. Jul. 10, 2003. Vol, 196, Issue 2, Pages 143-52.

Han, et al. 2′-Benzoyloxycinnamaldehyde induces Apoptosis in Human Carcinoma Via Reactive Oxygen Species. J. Biol. Chem. 2004. Feb. 20; 279:6911-20.

Bagul, et al. Crude Garlic Extract Inhibits Cell Proliferation and Induces Cell Cycle Arrest and Apoptosis of Cancer Cell in Vitro. J. Med. Food. 2015. July; 18(7):731-37.

Kim, et al. Hexane Extracts of Garlic Cloves Induce Apoptosis through the Generation of Reactive Oxygen Species in Hep3B Human Hepatocarcinoma Cells. Oncoogy Reports. Aug. 23, 2012. Pages: 1757-63.

Dozio, et al. The Natural Antioxidant Alpha-Lipoic Acid induces p27Kip1-Dependent Call Cycle Arrest and Apoptosis in MCF-7 Human Breast Cancer Cells. Euro. J. of Pharma. 2010. 641:29-34.

Moungjaroen, et al. Reactive Oxygen Species Mediate Caspase Activation and Apoptosis induced by Lipoic Acid in Human Lung Endothelial Cancer Cells through Bcl-2 Down-Regulation, J. of Pharma. And Exp. Thera. 2006. Dec.; 319(3):1062-69.

What is claimed is:

1. A chemo-suppression and chemo-protection composition, comprising:
an effective amount of a plurality of electronically modified oxygen derivative generating compounds, wherein the electronically modified oxygen derivative generating compounds are metabolized into a plurality of electronically modified oxygen derivatives which thereby induce a cellular apoptotic cascade within a population of tumorous or cancerous cells.

2. The composition of claim 1, wherein the electronically modified oxygen derivative generating compounds are selected from the group consisting artemisinin, superoxide dismutase, and glucose oxidase.

3. The composition of claim 2, further comprising electronically modified oxygen generating compounds selected from the group consisting of graviola, vitamin D3, turmeric, sulforaphane, cinnamaldehyde, garlic extract, and alpha lipoic acid.

4. The composition of claim 2, wherein the artemisinin comprises from about 100 milligrams to about 1,000 milligrams,

5. The composition of claim 2, wherein the superoxide dismutase comprises from about 50 milligrams to about 500 milligrams.

6. The composition of claim 2, wherein the glucose oxidase comprises from about 100 milligrams to about 1,000 milligrams.

7. The composition of claim 3, wherein the graviola comprises fromabout 100 milligrams to about 1,000 milligrams.

8. The composition of claim 3, wherein the vitamin D3 comprises from about 100 milligrams to about 2,000 milligrams.

9. The composition of claim 3, wherein the turmeric comprises from about 100 milligrams to about 1,000 milligrams.

10. The composition of claim 3, wherein the sulforaphane comprises from about 100 milligrams to about 1,000 milligrams.

11. The composition of claim 3, wherein the cinnamaldehyde comprises from 500 milligrams to about 2,000 milligrams.

12. The composition of claim 3, wherein the garlic extract comprises from about 1,000 milligrams to 10,000 milligrams.

13. The composition of claim 3, wherein the alpha lipoic acid comprises from about 300 milligrams to about 600 milligrams.

14. The composition of claim 1, wherein the electronically modified oxygen derivatives are selected from the group consisting of peroxides, superoxides, hydroxyl radicals, singlet oxygen, superoxide anions, and alkoxides.

15. The composition of claim 14, wherein the peroxide is hydrogen peroxide.

16. A method of inducing apoptosis in a population of cancer cells, the method comprising the steps of:
(a) administering a chemo-suppression composition comprising an effective amount of a plurality of electronically modified oxygen derivative generating compounds; and
(b) triggering at least one apoptotic enzyme by metabolizing the plurality of electronically modified oxygen derivative generating compounds, thereby generating electronically modified oxygen derivatives which thereby induce a cellular apoptotic cascade within a population of cancer cells.

17. The method of claim 16, wherein the triggering of apoptotic enzymes occurs within mitochondria of the population of cancer cells.

18. The method of claim 16, wherein the at least one apoptotic enzyme is caspase-9.
